(19) United States
(12) Patent Application Publication
(10) Pub. No.: US 2011/0082381 A1
(43) Pub. Date: Apr. 7, 2011

(54) Time Frequency Transformation Analysis for Detection and Quantification of Epileptiform Activity Load in Generalized Epilepsies

(76) Inventors: Basim M Uthman, Gainesville, FL (US); Panagote M. Pardalos, Gainesville, FL (US); Petros Xanthopoulos, Gainesville, FL (US); Chang-Chia Liu, Gainesville, FL (US); Steffen Rebennack, Gainesville, FL (US)

(21) Appl. No.: 12/866,591

(22) PCT Filed: Jun. 24, 2009
(86) PCT No.: PCT/US09/48488

§ 371 Date: Nov. 11, 2010

Related U.S. Application Data
(60) Provisional application No. 61/075,601, filed on Jun. 25, 2008.

(51) Int. Cl. A61B 5/0476

(52) U.S. Cl. 600/544

(57) ABSTRACT

Methods for detecting absence seizures are provided. An electroencephalogram (EEG) recording from a patient can be analyzed using an algorithm. The algorithm can include wavelet transform, in which wavelets can extract an original signal into scales that can be mapped into different pseudofrequencies. The algorithm can also include a sliding variance technique (SVT).

### CROSS-REFERENCE TO A RELATED APPLICATION

This application claims the benefit of U.S. provisional application Ser. No. 61/075,601, filed Jun. 25, 2008, which is incorporated herein by reference in its entirety.

### GOVERNMENT SUPPORT

The subject matter of this application has been supported by a research grant from the National Institute of Neurological Disorders and Stroke under grant number NINDS R01 NS046015 (KMK). Accordingly, the government has certain rights in this invention.

### BACKGROUND OF THE INVENTION

A seizure is a sudden loss of consciousness, a change in one's state of consciousness, and/or a loss of control over one's body. An absence seizure is one type of seizure which may occur in certain forms of epilepsy. Absence seizures are sometimes referred to as petit mal seizures. The term “petit mal” was originally coined by physicians and attendants in hospitals of Paris in the early 19th century, and the term “absence seizure” was introduced by Calmeil in 1824 (Da Silva, Electroencephalography: Basic Principles, Clinical Applications and Related Fields, Urban & Schwarzenberg, 1987).

The term petit mal underscores the decrease in convulsions normally associated with grand mal seizures. This contributes to the confusion between complex partial seizures (seizures of focal onset) and typical absence seizures (bihemispheric activity from onset). The two terms, petit mal and absence, can be complimentary; however, the latter may better describe symptoms of the seizures that can manifest as brief episodes of loss of consciousness and responsiveness. Absence seizures are generally short in duration, and it is not uncommon that they can be missed even by experienced witnesses such as parents and teachers. Furthermore, individuals with certain types of epilepsy can experience hundreds of absence seizures each day, resulting in poor performance at school and interfering with quality of life. Anti-epileptic drug (AED) treatments can help inhibit and control the occurrence of absence seizures (Schacther et al., The Comprehensive Evaluation and treatment of Epilepsy: A Practical Guide, Academic Press, 1997).

A common method for evaluating the efficacy of epilepsy treatments is to compare the number of seizures before treatment with the number of seizures after treatment during a finite period of time. To keep track of the number of seizures, the seizures experienced by patients and/or witnessed by observers can be documented by both parties in seizure diaries. However, such diaries have been found to be inaccurate as patients may not remember and observers may not recognize or be attentive to seizures at all times.

Absences seizures are often characterized by sudden loss of consciousness and/or interruption of motor activities for a brief period of time, which can last from a few seconds up to about thirty seconds. Given the short duration typically associated with absence seizures, along with their subtle clinical manifestations, absence seizures can be easily missed by inexperienced observers. In fact, even experienced observers can find it challenging to accurately evaluate and record occurrences of absence seizures.

In existing clinical trials, counting seizure frequency has traditionally been the method used most often in evaluating the efficacy of drug or other interventional therapy in the treatment of seizure disorders. This method tends to be quite tedious and plagued with several sources of measurement errors, including rater accuracy, rater and inter-rater reliability, experience of raters (usually family members or other companions of the patient) and erratic vigilance of observers. Due to the subtlety, high frequency, and short duration commonly associated with absence seizures, these factors are magnified, making the method of counting seizures by layman observers unreliable at best.

An electroencephalogram (EEG) is sometimes used to support the diagnosis of seizures and their types, but not typically for quantifying their occurrence. Manual evaluation of an acquired EEG and scoring of seizures by experienced electroencephalographers can sometimes be used. A typical absence seizure can be characterized by generalized and bilaterally synchronous spike and wave discharges (SWD), from about 5 seconds to about 20 seconds in duration. Like in most generalized epilepsies, SWD in absence seizures is maximal over the fronto-central midline and may start at a rate of about 4/sec, quickly slow down to about 3-3.5/sec, and during the final phase of the absence, slow to about 2.5/sec. FIG. 1 shows a time frequency spectrum of a typical absence seizure, and FIG. 2 shows an EEG recording of a typical absence seizure.

In some clinical studies of absence seizures, manual scoring of EEG recordings has been suggested for evaluating the efficacy of different AED treatments. The manual scoring of absence seizures is typically done by experienced qualified clinicians. However, this process is subject to the expertise and fatigue level of the clinician, as well as the possibility of manual recording errors. Additionally, this process is very time-consuming and tedious, sometimes taking several hours to score a few hours of EEG recordings. Therefore, manual EEG scoring can also be very expensive. Also, failure to spot seizures in an EEG recording can lead to misdiagnosis and even false evaluation of treatment effects.

Detection methods for absence seizures on EEG recording have been proposed in both human and animal models. Some methods utilize band pass filters to identify individual components or other amplitude duration criteria with or without filtering (Quy et al., High-speed Automatic Analysis of EEG Spike and Wave Activity Using an Analogue Detection and Microcomputer Plotting System, Electroencephalography and Clinical Neurophysiology, 49(1-2):187-9, July 1980; Carrie et al., Clinical Evaluation of a Method of Quantification of Generalized Spike-Wave EEG Patterns by Computer During Prolonged Recordings, Computers and Biomedical Research, 10:449-57, 1977; Principe et al., Microcomputer-based System for the Detection and Quantification of petit mal Epilepsy, Computers and Biomedical Research, 12:87-95, 1982; Koffler et al., Automatic Detection of Spike-and-Wave Bursts in Ambulatory EEG Recordings, Electroencephalography and Clinical Neurophysiology, 61(2):165-80, August 1985; Burr et al., Computerized Analysis of Epileptic Activity and Sleep in Mobile Long-Term EEG Monitoring, European Neurology, 25:61-65, 1986). In an animal model, an SWD detector was introduced based on the first derivative of EEG signals, called the steepness of the signals (Westerhuis et al., Automatic Detection of Spike-Wave Discharges in the Cortical EEG of Rats, Measuring Behavior '96, Int. Workshop on Methods and Techniques in Behavioral Research, trecht, The Netherlands, 16-18 Oct. 1996). However, this method sometimes misclassifies eye movement artifacts as absence seizures.

Other detection methods have been proposed, including a method based on the maximum absolute value of the EEG amplitude in the rat model (Fanselow et al., Reduction of Pentylenetetrazole-induced Seizure Activity in Awake Rats by Seizure-triggered Trigeminal Nerve Stimulation, The Journal of Neuroscience, 20:8160-8, 2000). However, this method cannot distinguish between high amplitude artifacts. A spectral-comb based analysis method has been proposed using a time frequency spectrum, produced by Short Time Fourier Transform (STFT), in order to extract some features that enable seizure detection (Van Hese et al., Detection of Spike and Wave Discharges in the Cortical EEG of Genetic Absence Epilepsy Rats from Strasbourg, Physics in Medicine and Biology, 48:1685-700, June 2003). Also, linear models and an artificial neural network has been used for attempting to detect absence seizures in a data set of several absence seizures acquired from patients (Alkan et al., Automatic Seizure Detection in EEG Using Logistic Regression and Artificial Neural Network, Journal of Neuroscience Methods, 148:167-176, 2005; Alkan et al., Comparison of Ar and Welch methods in Epileptic Seizure Detection, Journal of Medical Systems, 30:413-419, 2006). However, the performance of a neural network depends greatly on the training dataset. Thus, none of the existing detection methods is capable of quickly and accurately detecting absences seizures from an EEG recording.

### BRIEF SUMMARY OF THE INVENTION

The present invention provides novel methods for quickly and accurately detecting absence seizures.

Embodiments of the present invention provide novel methods for detecting and analyzing absence seizures using EEG recordings from patients.

In an embodiment, a method for detecting absence seizures from an EEG recording can include using wavelet transform. Wavelets can extract an original signal into scales that can be mapped into different pseudofrequencies. Wavelet transform can provide an advantage over Short Time Fourier Transform (STFT) since with wavelets, arbitrary resolution can be achieved in both time and frequency. A variance technique can also be applied to localize an absence seizure. The method can be performed using a processor.

In one embodiment, a method for detecting absence seizures from an EEG recording can include using an algorithm. The algorithm can be based, for example, on the spectral characteristics of the seizure. In a further embodiment, the algorithm can involve using wavelet transform, in which wavelets can extract an original signal into scales that can be mapped into different pseudofrequencies. In yet a further embodiment, the algorithm can include a sliding variance technique (SVT). A processor can be used to execute the algorithm.

When the phrase “a processor can be used to execute the algorithm” or “the algorithm is executed using a processor” is used herein, as a skilled artisan would understand, the meaning is that the steps of the algorithm can be performed using a processor a computer processor) and any calculations performed in the algorithm can be done by the processor.

### BRIEF DESCRIPTION OF THE DRAWINGS

FIG. 1 shows a time frequency plot for a typical absence seizure.

FIG. 2 shows an EEG recording for a typical absence seizure.

FIG. 3 shows a graphical representation of an algorithm according to an embodiment of the present invention.

FIG. 4 shows a Morlet mother wavelet function according to an embodiment of the present invention.

FIG. 5 shows an EEG recording of a false positive detected for a seizure free patient.

FIGS. 6A-6B show distributions of missed and detected epochs for a patient with absence seizures.

FIGS. 7A-7C show EEG recordings of missed seizures for a patient with absence seizures. Red lines indicate seizure onset and offset.

FIG. 8 shows electrode artifacts detected as SWD epochs.

FIG. 9 shows a graphical representation of Table 1.

FIG. 10A shows a sample of a generalized SWD recorded from a Fischer 344 rat.

FIG. 10B shows electrode placement on an animal.

FIG. 11 shows an absence seizure and scalogram prior to the seizure.

FIG. 12 shows a plot of sum of scales.

FIG. 13 shows a plot of variance values.

FIGS. 14A-14D show receiver operating characteristic curves.

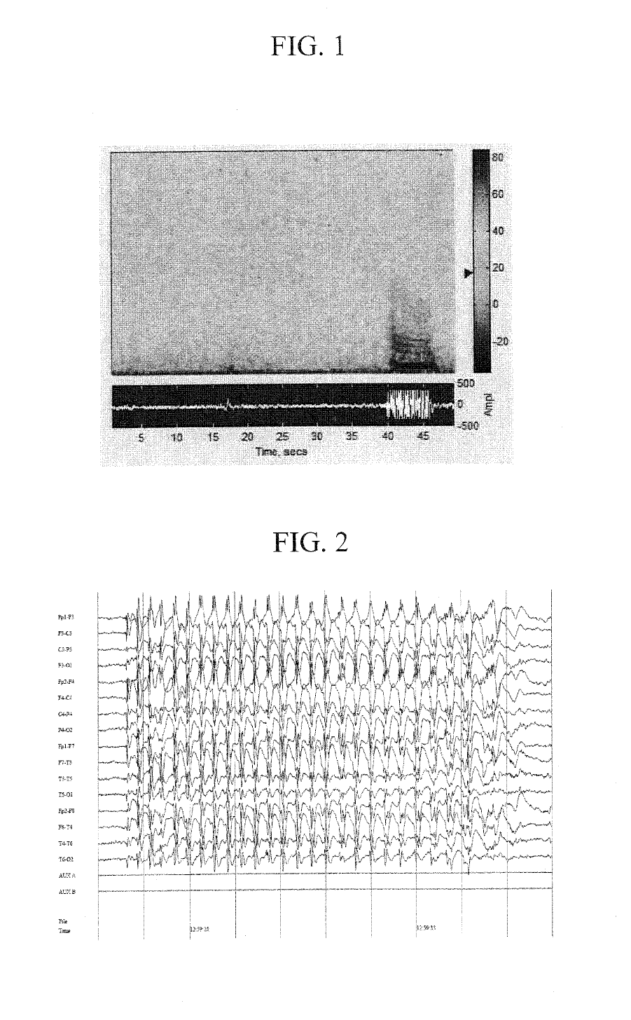

### DETAILED DESCRIPTION OF THE INVENTION

The present invention provides novel methods for quickly and accurately detecting absence seizures.

Generalized epilepsy (GE), primary or secondary, can often be associated with spike and/or polyspike and wave activity seen on an electroencephalogram (EEG) from onset to offset of seizures. Impairment of consciousness may be the initial manifestation and motor manifestations can be bilateral. Ictal electroencephalographic patterns can reflect neuronal discharge which may be widespread in both hemispheres of a patient's brain.

Ictal EEG characteristics of GE can include: regular, bilateral, and symmetrical spike-and-slow-wave complexes (can have multiple spike-and-slow-wave complexes for typical absence seizures); polyspike and wave, or sometimes spike and wave or sharp and slow waves for myoclonic seizures; and slow waves; fast activity occasional spike-and-wave patterns for clonic seizures; low voltage, fast activity or a fast rhythm of at least about 9 Hz, decreasing in frequency and increasing in amplitude for tonic seizures; fast rhythm of at least about 10 Hz at onset decreasing in frequency and increasing in amplitude during tonic phase, interrupted by slow waves during clonic phase for tonic-clonic seizures; polyspikes and wave or flattening or low-voltage fast activity for atonic seizures; and/or a decremental response for infantile spasms. Fast activity can refer to, for example, at least about 9 Hz.

The regular, bilateral, and symmetrical spike-and-slow-wave complexes can have a frequency of from about 2 Hz to about 4 Hz. Oftentimes, the frequency is about 3 Hz.

For atypical absence seizures, an EEG can sometimes have more heterogeneous, irregular, and slow spike-and-slow-wave complexes. Abnormalities can be bilateral and sometimes irregular and/or asymmetrical.

In absence seizures, an EEG can be used as a tool to measure and compare the quantity of seizure activity during a finite period of time before and during interventional therapy.

Embodiments of the present invention provide novel methods for detecting and analyzing absence seizures using EEG recordings from patients.

In an embodiment, a method for detecting absence seizures from an EEG recording can include using wavelet transform. Wavelets can extract an original signal into scales that can be mapped into different pseudofrequencies. Wavelet transform can provide an advantage over Short Time Fourier Transform (SIFT) since with wavelets, arbitrary resolution can be achieved in both time and frequency. A variance technique can also be applied to localize an absence seizure. The algorithm can be executed using a processor.

In one embodiment, a method for detecting absence seizures from an EEG recording can include using an algorithm. The algorithm can be based, for example, on the spectral characteristics of the seizure. In a further embodiment, the algorithm can involve using wavelet transform, in which wavelets can extract an original signal into scales that can be mapped into different pseudofrequencies. In yet a further embodiment, the algorithm can include a sliding variance technique (SVT). The algorithm can be executed using a processor.

When the phrase “a processor can be used to execute the algorithm” or “the algorithm is executed using a processor” is used herein, as a skilled artisan would understand, the meaning is that the steps of the algorithm can be performed using a processor (e.g., a computer processor) and any calculations performed in the algorithm can be done by the processor.

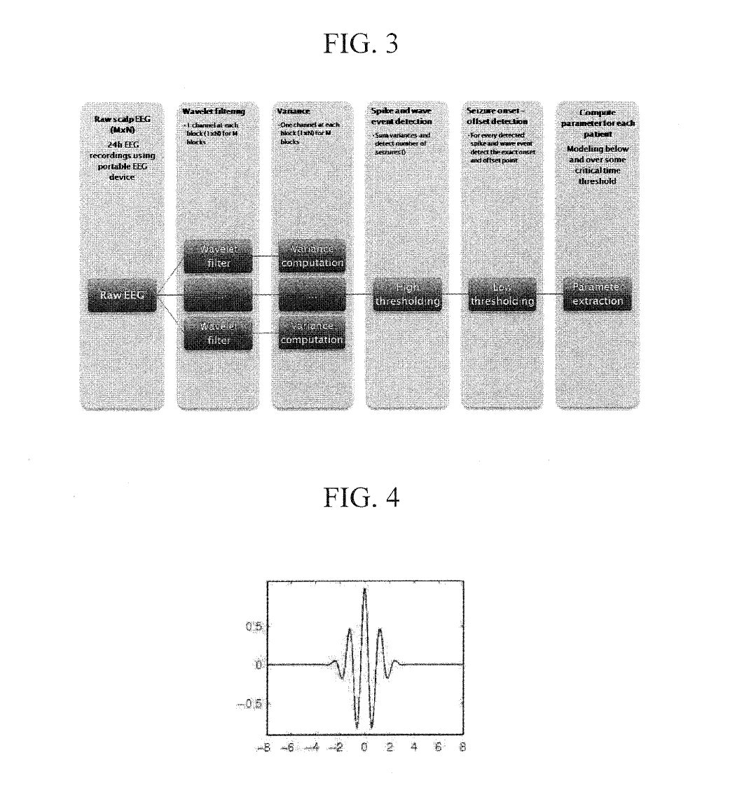

An algorithm according to an embodiment of the present invention can include computing the variance profile for each channel by using a moving window of length k samples. For each wavelet-filtered channel that can be seen as a time series of N sample points X=[x₁, X₁, . . . , x_{N}], the sample variances Vwᵢ=Var(wᵢ) can be computed that correspond to the sets wᵢ={xεX|x≧xᵢ, x<xᵢ₊₅} where i=1, . . . , N−k. After the variance calculation, the variance profiles for all channels can be added, and thresholding can be performed. Thresholding can be done since time intervals that contain seizure activity may have very high values of variance. Additionally, averaging can amplify characteristics common for all channels and help to cancel out noise. Thus, a series of consecutive ones in the indicator function 1{Vᵢ>P}, where P is some threshold, can suggest the presence of absence seizure activity. The first and the last 1 in such a series can correspond to seizure onset and offset, respectively. To inhibit the detection of artifacts that appear in a certain frequency band of interest, double-thresholding can be performed. That is, thresholding with a high threshold can be performed in order to detect a seizure, and then for each seizure detected local thresholding can be performed (with a lower threshold than the high threshold) in order to determine seizure onset and offset. The high threshold can be set as, for example, the maximum variance value during a seizure. The second threshold can be estimated from, for example, the variance value during the onset and the offset for known examples, if they exist. FIG. 3 shows a graphical representation of an algorithm according to an embodiment of the present invention. In an embodiment, the algorithm can be executed using a processor.

In a specific embodiment, a method for detecting absence seizures from an EEG recording can include using an algorithm with the pseudocode given in algorithm (1) below.

Algorithm (1):
Require: EEG recording X^{m} with N sample points for each M channels in Parameters: high threshold, low threshold, sample size k
Ensure: onset and offset of all detected seizures
1: Continuous Wavelet Transformation:
2: for all channel m do
3: call Y^{m}=CWTA(Xₘ, α_{low}, α_{high}, β)
4: end for
5: Variance Computation:
6: for all channel m do
7: for time windows i with k samples do
8: compute variance vᵢ^{m}
9: end for
10: end for
11: sum the variances: vᵢ=Σₘ vᵢ^{m}
12: Spike and Wave Discharge (SWD) Epoch Detection:
13: for vᵢ≧high threshold do
14: onset=closest right point j where vⱼ≦low threshold
15: offset=closest left point j where vⱼ≧low threshold
16: end for
17: Merge SWD Epochs with a distance of less than 1 sec.
18: return all detected SWD Epochs.

The Function CWTA(X^{m}, α_{low}, α_{high}, β) can be, for example, a function with the pseudocode of Function (1) below.

Function (1):
1: for j=α_{low} to j=α_{high} with step β do
2: compute Yⱼ with Formula (A) for j
3: end for
4: return Y^{m}=Σⱼ Yⱼ.

The Formula (A) used in Function (1) can be a wavelet transform formula. For example, Formula (A) can be the Continuous Wavelet Transform (CWT) formula of Equation (1) below.

C(t,a,b)=∫_{-∞}^{+∞}x(t)ψ_{a,b}(τ)dτ (1):

where

$${ψ}_{a , b} ( τ ) = \frac{1}{\sqrt{α}} ψ ( \frac{τ - b}{a} )$$

is the mother wavelet function. The mother wavelet function can be the Morlet wavelet with an analytic expression given by

$$ψ ( τ ) = {e}^{\frac{{τ}^{2}}{2}} cos ( 5 τ ) .$$

FIG. 4 shows a graphical depiction of a Morlet wavelet function. Though Formula (A) of Function (1) has been described as Equation (1), embodiments of the present invention are not limited thereto.

Additionally, though line 17 of the pseudocode of Algorithm (1) indicates that spike and wave discharge (SWD) epochs with a distance of less than 1 sec apart should be merged, embodiments of the present invention are not limited to this time value. Any suitable time value can be used for the limit for merging SWD epochs. For example, in an embodiment, SWD epochs that are less than about 0.5 seconds apart can be merged and counted as one SWD epoch. In another embodiment, SWD epochs that are less than about 1.5 seconds apart can be merged and counted as one SWD epoch. In a further embodiment, SWD epochs that are less than about 0.75 seconds apart can be merged and counted as one SWD epoch. In yet a further embodiment, SWD epochs that are less than about 0.25 seconds apart can be merged and counted as one SWD epoch. In yet a further embodiment, SWD epochs that are less than about 2 seconds apart can be merged and counted as one SWD epoch. In yet a further embodiment, SWD epochs that are less than about 2.5 seconds apart can be merged and counted as one SWD epoch. In yet a further embodiment, SWD epochs that are less than about 3 seconds apart can be merged and counted as one SWD epoch. In yet a further embodiment, SWD epochs that are less than about 1.25 seconds apart can be merged and counted as one SWD epoch.

One advantage of wavelet transform over SIFT analysis is that frequency resolution can be increased in certain frequency bands while maintaining approximately the same time resolution. For example, wavelet transform can give higher frequency resolution than STFT in the delta band at 3 Hz. This can be especially useful in SWDs of absences seizures because these discharges often occur in a frequency window of from about 2.5 Hz to about 4.5 Hz.

With wavelet transform, scales can be transformed into frequencies using Equation (2).

Fₐ=F_{c}/(αΔ) (2):

where F_{α} is the frequency that corresponds to the scale α, F_{c} is the central mother wavelet frequency, and Δ is the EEG sampling period. In a specific embodiment of the present invention using the Morlet mother wavelet function, F_{c} can be about 0.81 Hz and Δ can be about 1/200 sec.

Additionally, in a certain embodiment, the sampling frequency can be fₛ=200 Hz, M can be 16 channels, and the scales 36:46 can be used, corresponding to the frequencies 2.5 Hz to 4.5 Hz. In this context, wavelet transform can be used as a band pass filter by keeping scales of interest and rejecting other scales.

### EXEMPLIFIED EMBODIMENTS

### Embodiment 1

A method for detecting absence seizures, comprising:
performing an algorithm including wavelet transform on an electroencephalogram (EEG) recording of a patient.

### Embodiment 2

The method according to embodiment 1, wherein the algorithm comprises a sliding variance technique (SVT).

### Embodiment 3

The method according to any of embodiments 1-2, wherein the algorithm comprises computing a variance profile by using a moving window with a length given by a number of samples.

### Embodiment 4

The method according to any of embodiments 1-3, wherein the algorithm further comprises performing a thresholding process.

### Embodiment 5

The method according to any of embodiments 1-4, wherein the algorithm further comprises performing a double-thresholding process.

### Embodiment 6

The method according to any of embodiments 1-5, wherein the algorithm has a pseudocode of Algorithm (1):

Algorithm (1):
Require: EEG recording X^{m} with N sample points for each M channels m Parameters: high threshold, low threshold, sample size k
Ensure: onset and offset of all detected seizures
1: Continuous Wavelet Transformation:
2: for all channel m do
3: call Y^{m}=CWTA(X^{m}, α_{low}, α_{high}, β)
4: end for
5: Variance Computation:
6: for all channel m do
7: for time windows i with k samples do
8: compute variance vᵢ^{m}
9: end for
10: end for
11: sum the variances: vᵢ=Σₘ vᵢ^{m}
12: Spike and Wave Discharge (SWD) Epoch Detection:
13: for vᵢ≧high threshold do
14: onset=closest right point j where vⱼ≦low threshold
15: offset=closest left point j where vⱼ≦low threshold
16: end for
17: Merge SWD Epochs with a distance of less than T
18: return all detected SWD Epochs.

### Embodiment 7

The method according to embodiment 6, wherein T in line 17 of Algorithm (1) is about 1 second.

### Embodiment 8

The method according to any of embodiments 6-7, wherein CWTA(X^{m}, α_{low}, α_{high}, β) is a function with the pseudocode of Function (1) below.

Function (1):
1: for j=α_{low} to j=α_{high} with step β do
2: compute Yⱼ with Formula (A) for j
3: end for
4: return Y^{m}=Σⱼ Yⱼ.

### Embodiment 9

The method according to embodiment 8, wherein Formula (A) is the Continuous Wavelet Transform (CWT) formula of Equation (1) below.

C(t,a,b)=∫_{-∞}^{+∞}x(t)ω_{a,b}(τ)dτ Equation (1):

where

$${ψ}_{a , b} ( τ ) = \frac{1}{\sqrt{α}} ψ ( \frac{τ - b}{a} )$$

is the mother wavelet function.

### Embodiment 10

The method according to embodiment 9, wherein the mother wavelet function is the Morlet wavelet function with the analytic expression

$$ψ ( τ ) = {e}^{\frac{{τ}^{2}}{2}} cos ( 5 τ ) .$$

### Embodiment 11

A method for detecting absence seizures, comprising performing an algorithm on an EEG recording of a patient, wherein the algorithm has a pseudocode of Algorithm (1):

Algorithm (1):
Require: EEG recording X^{m} with N sample points for each M channels m Parameters: high threshold, low threshold, sample size k
Ensure: onset and offset of all detected seizures
1: Continuous Wavelet Transformation:
2: for all channel m do
3: call Y^{m}=CWTA(X^{m}, α_{low}, α_{high}, β)
4: end for
5: Variance Computation:
6: for all channel m do
7: for time windows i with k samples do
8: compute variance vᵢ^{m}
9: end for
10: end for
11: sum the variances: vᵢ=Σₘ vᵢ^{m}
12: Spike and Wave Discharge (SWD) Epoch Detection:
13: for vᵢ≧high threshold do
14: onset=closest right point j where vⱼ≦low threshold
15: offset=closest left point j where vⱼ≦low threshold
16: end for
17: Merge SWD Epochs with a distance of less than T
18: return all detected SWD Epochs.

### Embodiment 12

The method according to embodiment 11, wherein T in line 17 of Algorithm (1) is about 1 second.

### Embodiment 13

The method according to any of embodiments 11-12, wherein CWTA(X^{m}, α_{low}, α_{high}, β) is a function with the pseudocode of Function (1) below.

Function (1):
1: for j=α_{low} to j=α_{high} with step β do
2: compute Yⱼ with Formula (A) for j
3: end for
4: return Y^{m}=Σⱼ Yⱼ.

### Embodiment 14

The method according to embodiment 13, wherein Formula (A) is the Continuous Wavelet Transform (CWT) formula of Equation (1) below.

C(t,a,b)=∫_{-∞}^{+∞}x(t)ω_{a,b}(τ)dτ Equation (1):

where

$${ψ}_{a , b} ( τ ) = \frac{1}{\sqrt{α}} ψ ( \frac{τ - b}{a} )$$

is the mother wavelet function.

### Embodiment 15

The method according to embodiment 14, wherein the mother wavelet function is the Morlet wavelet function with the analytic expression

$$ψ ( τ ) = {e}^{\frac{{τ}^{2}}{2}} cos ( 5 τ ) .$$

### Embodiment 16

The method according to any of embodiments 1-10, wherein the algorithm is executed using a processor.

### Embodiment 17

The method according to any of embodiments 11-15, wherein the algorithm is executed using a processor.

### EXAMPLES

### Example 1

A study was conducted to measure absence seizures from patients using EEG recordings. Two patients were included in the study; one seizure free and one experiencing over 100 seizures within 4.5 hours. Ambulatory EEG recordings were acquired from two children <13 years of age; one seizure free for 24 hours and one experiencing seizures within 4.5 hours. Subjects were instructed to go about their normal life as usual while EEG recording was ongoing avoiding any type of activity that might result in the loosening or removal of electrodes from the scalp or result in excessive recording artifacts, e.g., gum chewing. The international 10-20 electrode placement system with 19 electrodes was used and the following 16 bipolar channels were chosen: Fp1-F3, F3-C3, C3-P3, P3-01, Fp2-F4, F4-C4, C4-P4, P4-02, Fp1-F7, F7-T3, T3-T5, T5-O1, Fp2-F8, F8-T4, T4-T6, T6-O2. Data points were collected at a sampling rate of 200 Hz for each channel. EEG recordings were scored by a clinically experienced board certified electroencephalographer noting the duration of each SWD from onset to offset to one decimal point of a second. Operationally, two separate epochs of SWD complexes were counted as one event when the inter-epoch duration was less than about 1 second.

An algorithm having the pseudocode of Algorithm (1) was used and detected only one false positive finding in the first patient and detected 120 out of 150 continuous uninterrupted 3 Hz spike SWD epochs in the second patient. Of the 30 SWD epochs missed in the second patient, 27 were less than 2.1 seconds in duration. The remaining epochs were 3.1 seconds, 3.3 seconds, and 4.1 seconds of interrupted 3 Hz SWDs. The algorithm used provided an efficient., automatic detection scheme for diagnostic and therapeutic evaluation in patients with absence seizures.

Additionally, an electroencephalographer labeled and scored all SWD epochs separately and independently from the algorithm. The onset and offset of each epoch of continuous 3 Hz SWD were recorded to the nearest first decimal utilizing digital time stamped by the EEG acquisition machine. Any typical 3 Hz SWD interrupted by less than a one second interval was defined as one epoch of 3 Hz SWD. Onset was subtracted from offset times to obtain duration of each epoch in seconds. Durations of all epochs obtained by manual scoring were compared to durations of matching epochs detected by the algorithm.

A false positive was defined as an artifact or EEG misclassified as a seizure, a false negative was defined as an SWD epoch that was not detected by the algorithm. Occurrences of false positives, total time of false negatives, and precision in detecting seizure onset and offset time were considered.

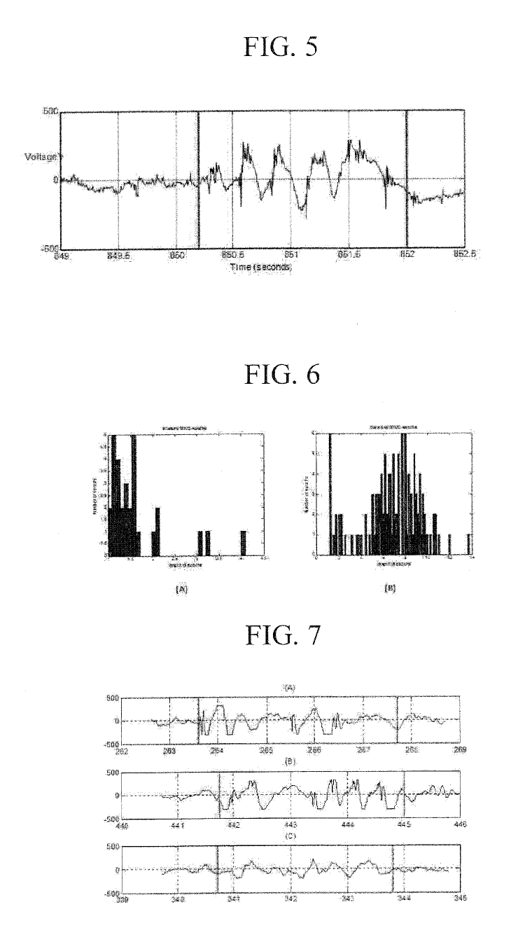

For patient 1 with 24 hours of seizure free EEG recordings, the algorithm produced only one false positive detection. This false positive is shown in FIG. 5. Multiple sources of false positivity such as chewing artifacts, eye movement artifacts, vertex waves, sleep spindles, and others occurred frequently during the 24 hour recording analyzed. However, the algorithm rejected all these artifacts and reported only one false positive epoch that was 2 seconds in duration. This epoch was reexamined by the electroencephalographer and confirmed to be an artifact.

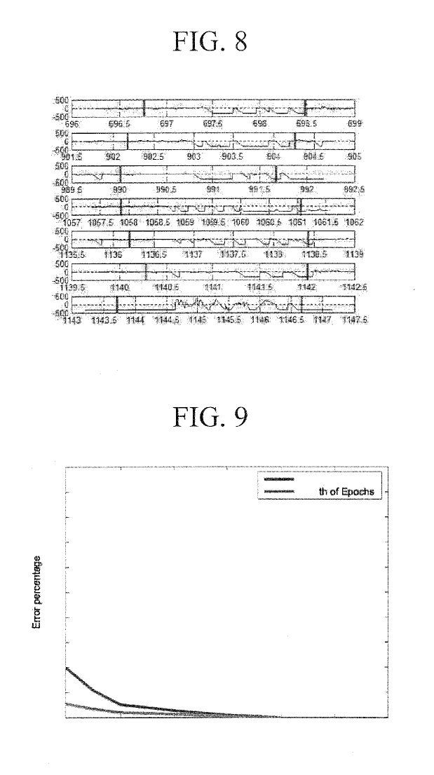

Of 150 manually scored 3 Hz SWD epochs in patient 2, the algorithm detected 120. The distribution of missed and detected 3 Hz SWD epochs by duration is shown in FIGS. 6A and 6B, respectively. Twenty-seven of the 30 missed epochs were less than 2.1 seconds in duration. The remaining epochs were 3.1 seconds, 3.3 seconds, and 4.1 seconds, and are shown in FIGS. 7A, 7B, and 7C, respectively. All 3 missed epochs longer than 3 seconds turned out to be fragmented 3 Hz SWD with interruptions of less than 1 second; episodes that were defined operationally as one epoch of 3 Hz SWD. In this patient 2 data set of 4.5 hours of EEG recordings, the algorithm detected a total of 7 false positive epochs, shown in FIG. 8.

The sliding window length chosen helps determine how well the algorithm detects short epochs. Smaller windows can be very sensitive to small changes in frequency and amplitude but can also be contaminated by artifacts of similar changes. On the other hand, if the variance window becomes too long (in samples), it can be easy to miss epochs shorter than the window length. A 1 second window was chosen due to the fact that SWD epochs 3 sec long are not generally clinically important. The algorithm detected successfully 120 3 Hz SWD epochs. In total, the percentage of error in terms of number of 3 Hz SWD epochs was 20% ( 30/150), as calculated by Equation (3).

% Error=(#missed/# manually scored)×100% (3):

The percentage error in terms of cumulative time of missed 3 Hz SWD time was 5.61% (48.82 sec/870.60 sec), as calculated by Equation (4).

% Error=(cumulative time missed/cumulative manually scored time)×100% (4):

The fact that the second error percentage was very low means that the majority of missed epochs were of short length. Table 1 and FIG. 9 depict the percentage error based on number of missed 3 Hz SWD epochs and duration of missed 3 Hz SWD epochs by duration of epochs.

TABLE 1
Percentage Error as a Function of SWD Epoch.

| | Epoch > 1 | E > 1.5 | E > 2 | E > 3 | E > 4 | E > 5 |
|---|---|---|---|---|---|---|
| | sec | sec | sec | sec | sec | sec |
| % error | 20.00% | 10.94% | 5.13% | 2.75% | 0.97% | 0.00% |
| based on # | | | | | | |
| missed | | | | | | |
| % error | 5.61% | 3.51% | 2.04% | 1.31% | 52.00% | 0.00% |
| based on | | | | | | |
| duration | | | | | | |
| missed | | | | | | |

In Table 1, the percentage error based on # missed is calculated from Equation (3) considering only the epochs longer than x seconds in each column (where x=1 in the first column, 1.5 in the second column, etc.). The percentage error based on duration missed is calculated from Equation (4) considering only the epochs longer than x seconds in each column (where x=1 in the first column, 1.5 in the second column, etc.).

For the successfully detected events the error was computed both in terms of number of samples and duration (seconds) for the onset, and offset of SWD. The results are shown in Table 2. Also, Table 3 shows the error for the total detected duration.

TABLE 2
Error for Onset and Offset for Detected Seizures

| | Error # | Error time | Error # | Error time |
|---|---|---|---|---|
| | samples | (sec) | samples | (sec) |
| Mean | 66.48 | 0.33 | 81.94 | 0.41 |
| Std Dev | 60.7 | 0.3 | 101.13 | 0.51 |

TABLE 3
Error for the Total Duration of Detected Seizures

| | Error # | Error # |
|---|---|---|
| | samples | samples |
| Mean | 65.74 | 0.33 |
| Std Dev | 92.62 | 0.46 |

False positives detected in patient 2 are shown in FIG. 8. In total, seven false positives were detected. These false positives were due to high amplitude electrode artifacts. On average, the mean duration of the detected artifacts was 2.22 seconds with standard deviation of 0.62 seconds.

Despite the difficulty in detecting absence seizure, the algorithm detected clinically significant 3 Hz SWD epochs with high sensitivity and precision. Only one false positive epoch in patient 1 was detected and 97.25% of all 3 Hz SWD about 3 sec long were detected in patient 2.

The sensitivity of the proposed algorithm is influenced by the length of the sliding window. Smaller window length can lead to higher sensitivity and higher chance for false positive detection while larger window length can result in lower resolution and lower chance for false positive detection. The window length in this example was chosen to yield high sensitivity and specificity for the 3 Hz SWD.

### Example 2

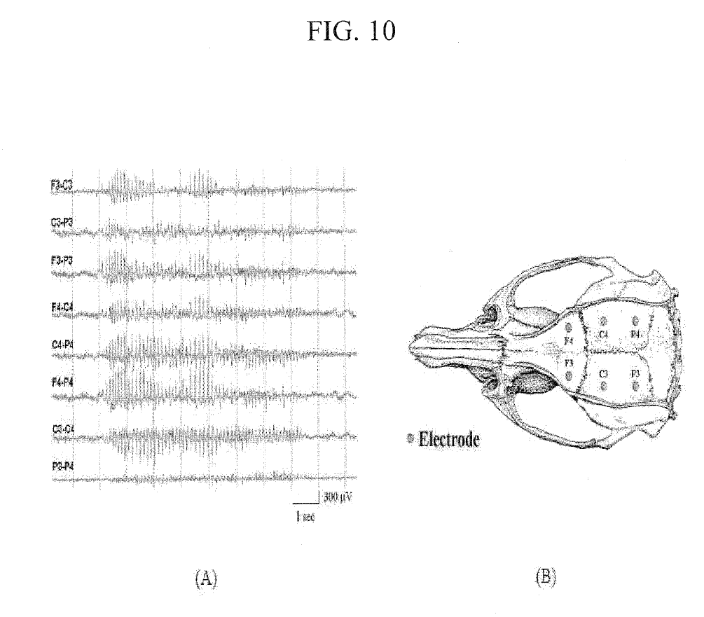

A study was conducted using the algorithms of the subject invention on animal models. Eight-hour recordings were acquired from a total of four 4-month old Fischer 344 (F344) rats. In total, six screw electrodes were implanted in the skull of each animal: 2 frontal (F3, F4), 2 central (C3, C4), and 2 parietal (P3, P4). The electrode configuration is shown in FIG. 10B. In total, eight differential channels were computed for the purpose of the study: F3-C3, C3-P3, F3-P3, F4-C4, C4-P4, F4-P4, C3-C4, and P3-P4. FIG. 10A shows a sample of a generalized SWD recorded from an F344 rat. The F3, C3, and P3 abbreviations refer to skull screw electrodes overlying left frontal, central, and parietal regions of the animal's brain, respectively; F4, C4, and P4 refer to the brain areas on the right. An “F3-C3” label corresponds to an EEG channel produced by the output of one differential amplifier with inputs from the F3 and C3 electrodes.

Long term video-EEG recordings were visually scanned to detect and score SWD occurrence; identified SWDs were confirmed by an electroencephalographer. The exact number of epochs and their cumulative time during the 8 hours of recordings can be seen in the Table 4.

TABLE 4
Number of SWD Epochs Scored for Each Rat and the
Cumulative Time of SWDs During the Recordings

| | Number of | Cumulative ictal | Total recording |
|---|---|---|---|
| | SWD epochs | activity (epochs) | time (hrs) |
| Rat A | 53 | 99.33 | 8.27 |
| Rat B | 43 | 116.35 | 8.09 |
| Rat C | 81 | 368.53 | 8.00 |
| Rat D | 45 | 133.50 | 8.10 |
| Total | 222 | 717.71 | 32.46 |

For detecting SWD discharges in a rat model, the proposed detection scheme is based in time frequency decomposition of the EEG employing the wavelet transform. Wavelet transform has profound advantages over the classical STFT because one can increase the scale (or frequency) resolution while keeping the same time resolution. Subsequently, the variance profile of the EEG is computed and seizures are detected by a double thresholding process. The algorithm was found to have high sensitivity and a minimal false positive detection rate for SWDs localized in the frequency band of about 3 Hz. For the detection of SWDs, an algorithm was used for detection including wavelet decomposition, variance profile computation, and thresholding.

Every differential channel of the raw EEG recordings, which can be represented as a time series X(t), was decomposed into a time-scale domain using the wavelet transform: C(t, a, b)=Σ_{-∞}^{∞}x(t)ψ_{a,b}(τ)dτ, where

$${ψ}_{a , b} ( τ ) = \frac{1}{\sqrt{α}} ψ ( \frac{τ - b}{a} )$$

is the mother wavelet function. ψ(τ)=cos(5τ) was used, which is a form of the Morlet mother wavelet. The Morlet mother wavelet has a low time-bandwidth product, infinite differentiation, and explicit expression, which are useful in EEG analysis. A time scale plot of a recorded absence seizure is shown in FIG. 11.

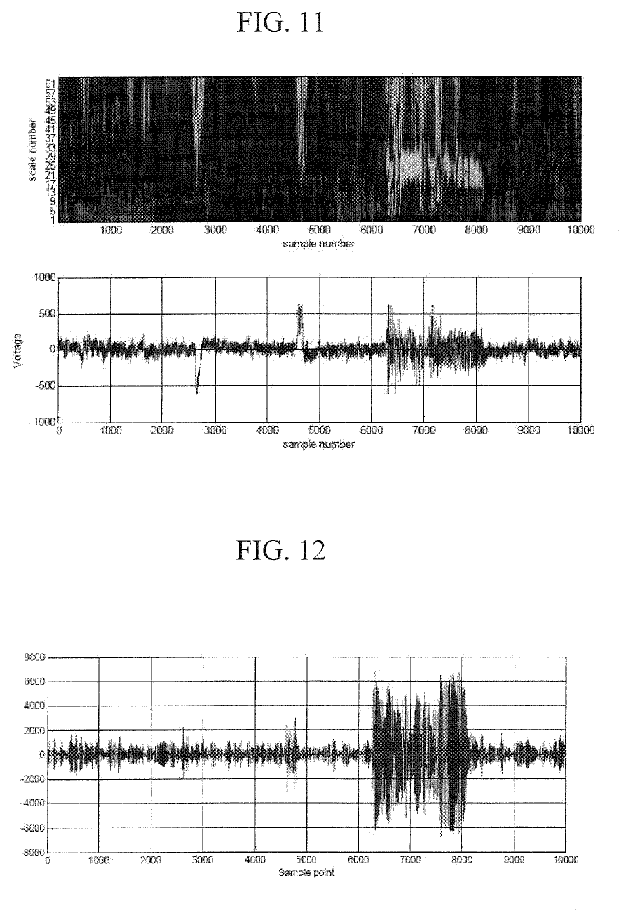

Scales were converted into frequencies using

$${f}_{a} = \frac{{f}_{c}}{αΔ} ,$$

where f_{c} is the central frequency of the mother wavelet, in this case, 0.81 Hz, and Δ= 1/200 sec is the sampling period. Among all the scales that can decompose the EEG signal, most important are those that correspond to the frequency band in which SWD activity appears (for example, about 7 Hz). Thus, only the scales 19-25 were kept and summed for every channel. FIG. 12 shows a plot of these sums.

Based on the observation that the high SWD activity produces wavelet profiles of high variance, the variance profile was computed for each channel using a sliding window of width k=200 samples (i.e., corresponding to 1 sec of recording). All variance profiles for all channels were summed to reject noise and artifacts that are not generalized (i.e., not appearing in all channels). A variance profile of the seizure can be seen in FIG. 13.

For accurate localization of the onset and offset times of a seizure based on the variance profile of the EEG recordings, a double thresholding technique was used. The two thresholds can be seen as the flat lines in FIG. 13. A high threshold was applied to the variance to detect the number of epochs; the high threshold was chosen, in part, to avoid detection of artifacts (false positives).

For the sample points of the variance profile curve that “hit” the high threshold, a local search was performed to specify the exact onset and offset sample points of the seizure. That is, referring to FIG. 13, for the first point that corresponds to an offset (first point that high line hits the variance profile), a backward search was performed to determine the first time that the variance curve falls below the low threshold (first point that low threshold intersects with variance profile). For the second point, a forward search was performed to determine the first point that the variance curve drops below the low threshold. For the third and fourth points, the same search process was repeated (third point will correspond again to an onset and the fourth point to an offset). With the low thresholding search, epochs that were detected as two distinct events from the high thresholding can be merged (i.e., in this example the onset and the offset of both epochs will be the same). The algorithm returns only the unique events; duplicates are rejected.

The detection sensitivity and false positive rate can be highly dependent on the parameters of the algorithm (e.g., thresholds). The algorithm was applied using 10 different thresholds, and sensitivity and specificity were computed. Sensitivity was defined as the number of epochs detected over the total number of scored epochs, and false positive rate was defined as the number of false positives over the corresponding recording time.

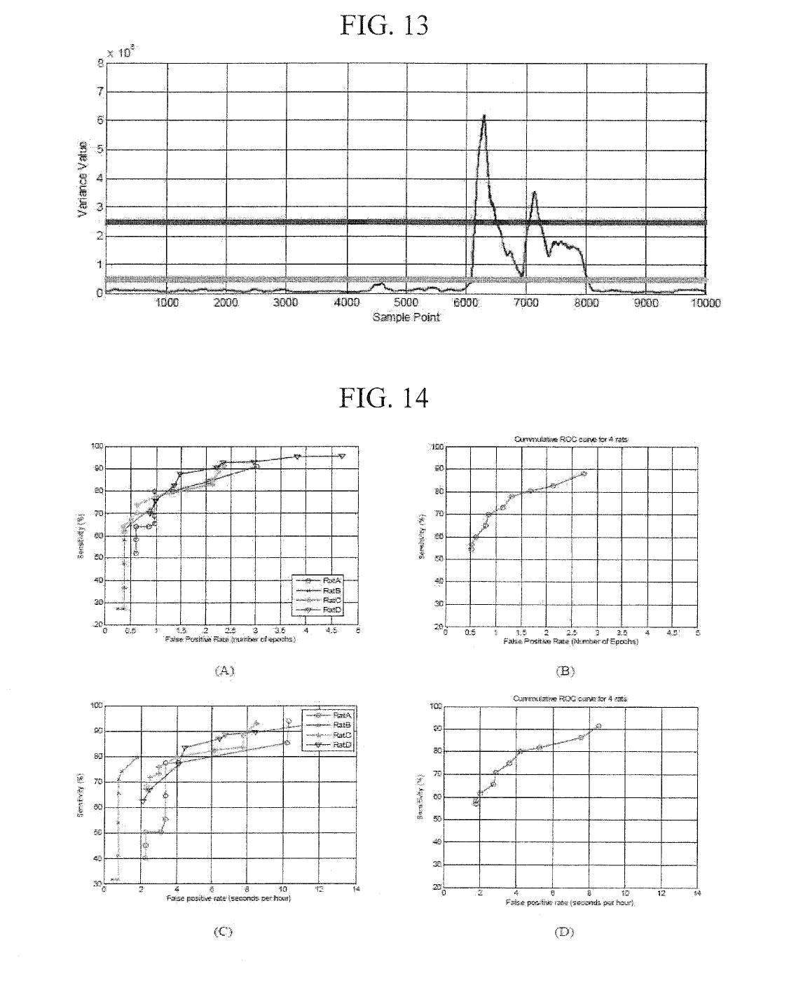

A receiver operating characteristic (ROC) curve can be used as a plot of sensitivity versus specificity. The ROC can be very useful in visualizing how the sensitivity percentage changes as a function of missed seizure rate and help an end user to decide the optimal point (corresponding threshold) that fits a specific application. FIGS. 14A-14D show ROC curves obtained. FIGS. 14A and 14B show the ROC curves for the four rats separately and the mean curve for all four rats, respectively.

With this definition of sensitivity and false positive rate, all epochs are treated in the same manner without taking into consideration the epoch length. That is, one SWD epoch of 10 sec in length will contribute the same as an epoch of 1 sec. Given this consideration, sensitivity and specificity can be defined based on the cumulative SWD time or “cumulative epileptiform burden.” In this case, sensitivity is defined as the cumulative detected time (in see) over the total time, whereas the false positive rate is defined as the cumulative missed seizure time over the corresponding time of the recordings.

FIGS. 14C and 14D show the ROC curves for the four rats using length of epochs and the mean curve for all four rats using length of epochs, respectively. The fact that the ROC curve using the cumulative time (FIG. 14B) has higher sensitivity values compared to that with the detected number of epochs (FIG. 14D) means that the missed epochs are shorter compared to the detected epochs. SWD epochs of all lengths were considered in this study, though in clinical practice, a frequently encountered issue is whether SWDs are sufficiently long to result in an absence seizure (e.g., a 0.5 sec SWD epoch would not be clinically significant). Under such assumptions, the detection sensitivity and false positive rate would improve drastically. Therefore, the error analysis presented here can be viewed as an upper limit of the error range. The second parameter of the algorithm (low threshold) can help determine the accuracy of the seizure onset and offset detection. Referring to FIGS. 14A-14D, changes in the low threshold can modify the onset and offset detection point by some number of sample points, which correspond to 1/200 sec each.

The proposed algorithm is robust with respect to the input parameters, which means that the output (detected epochs) cannot differ significantly when small changes are made to the threshold parameters. In addition, the ROC analysis showed that high sensitivity rates (greater than about 90%) can be achieved along with a low false positive rate (about 2 to about 4 false positive epochs per hour). When considering the cumulative time of the SWD epochs instead of the absolute number of the epochs, the algorithm demonstrated, on average, over 90% accuracy while the total missed SWD time did not exceed about 8.5 seconds per hour. The sensitivity percentages and the false positive rates can increase dramatically when epochs longer than some predefined duration are considered.

All patents, patent applications, provisional applications, and publications referred to or cited herein, supra or infra, are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

What is claimed is:

1. A method for detecting absence seizures, comprising:
performing an algorithm including wavelet transform on an electroencephalogram (EEG) recording of a patient.

2. The method according to claim 1, wherein the algorithm comprises a sliding variance technique (SVT).

3. The method according to claim 1, wherein the algorithm comprises computing a variance profile by using a moving window with a length given by a number of samples.

4. The method according to claim 3, wherein the algorithm further comprises performing a thresholding process.

5. The method according to claim 3, wherein the algorithm further comprises performing a double-thresholding process.

6. The method according to claim 1, wherein the algorithm has a pseudocode of Algorithm (1):
Algorithm (1):
Require: EEG recording X^{m} with N sample points for each M channels m Parameters: high threshold, low threshold, sample size k
Ensure: onset and offset of all detected seizures
1: Continuous Wavelet Transformation:
2: for all channel m do
3: call Y^{m}=CWTA(X^{m}, α_{low}, α_{high}, β)
4: end for
5: Variance Computation:
6: for all channel m do
7: for time windows i with k samples do
8: compute variance vᵢ^{m}
9: end for
10: end for
11: sum the variances: vᵢ=Σₘ vᵢ^{m}
12: Spike and Wave Discharge (SWD) Epoch Detection:
13: for vᵢ≧high threshold do
14: onset=closest right point j where vⱼ≦low threshold
15: offset=closest left point j where vⱼ≦low threshold
16: end for
17: Merge SWD Epochs with a distance of less than T
18: return all detected SWD Epochs.

7. The method according to claim 6, wherein T in line 17 of Algorithm (1) is about 1 second.

8. The method according to claim 6, wherein CWTA(X^{m}, α_{low}, α_{high}, β) is a function with the pseudocode of Function (1) below.
Function (1):
1: for j=α_{low} to α_{high} with step β do
2: compute Yⱼ with Formula (A) for j
3: end for
4: return Y^{m}=Σⱼ Y^{j}.

9. The method according to claim 8, wherein the algorithm is executed using a processor.

10. The method according to claim 8, wherein Formula (A) is the Continuous Wavelet Transform (CWT) formula of Equation (1) below.
C(t,a,b)=∫_{-∞}^{+∞}x(t)ω_{a,b}(τ)dτ Equation (1):
where
$${ψ}_{a , b} ( τ ) = \frac{1}{\sqrt{α}} ψ ( \frac{τ - b}{a} )$$
is the mother wavelet function.

11. The method according to claim 10, wherein the mother wavelet function is the Morlet wavelet function with the analytic expression
$$ψ ( τ ) = {e}^{\frac{{τ}^{2}}{2}} cos ( 5 τ ) .$$

12. A method for detecting absence seizures, comprising performing an algorithm on an EEG recording of a patient, wherein the algorithm has a pseudocode of Algorithm (1):
Algorithm (1):
Require: EEG recording X^{m} with N sample points for each M channels in Parameters: high threshold, low threshold, sample size k
Ensure: onset and offset of all detected seizures
1: Continuous Wavelet Transformation:
2: for all channel m do
3: call Y^{m}=CWTA(X^{m}, α_{low}, α_{high}, β)
4: end for
5: Variance Computation:
6: for all channel m do
7: for time windows i with k samples do
8: compute variance vᵢ^{m}
9: end for
10: end for
11: sum the variances: vᵢ=Σₘ vᵢ^{m}
12: Spike and Wave Discharge (SWD) Epoch Detection:
13: for vᵢ≧high threshold do
14: onset=closest right point j where vⱼ≦low threshold
15: offset=closest left point j where vⱼ≦low threshold
16: end for
17: Merge SWD Epochs with a distance of less than T
18: return all detected SWD Epochs.

13. The method according to claim 12, wherein T in line 17 of Algorithm (1) is about 1 second.

14. The method according to claim 12, wherein CWTA(X^{m}, α_{low}, α_{high}, β) is a function with the pseudocode of Function (1) below.
Function (1):
1: for j=α_{low} to j=α_{high} with step β do
2: compute Yⱼ with Formula (A) for j
3: end for
4: return Y^{m}=Σⱼ Yⱼ.

15. The method according to claim 14, wherein the algorithm is executed using a processor.

16. The method according to claim 14, wherein Formula (A) is the Continuous Wavelet Transform (CWT) formula of Equation (1) below.
C(t,a,b)=∫_{-∞}^{+∞}x(t)ω_{a,b}(τ)dτ Equation (1):
where
$${ψ}_{a , b} ( τ ) = \frac{1}{\sqrt{α}} ψ ( \frac{τ - b}{a} )$$
is the mother wavelet function.

17. The method according to claim 16, wherein the mother wavelet function is the Morlet wavelet function with the analytic expression
$$ψ ( τ ) = {e}^{\frac{{τ}^{2}}{2}} cos ( 5 τ ) .$$
